(19) United States
(12) Patent Application Publication
(10) Pub. No.: US 2008/0003275 A1
(43) Pub. Date: Jan. 3, 2008

(54) Treatment of Premature Ejaculation

(75) Inventor: Jakov Vaisman, Bondi Junction (AU)

Correspondence Address: EDWARDS ANGELL PALMER & DODGE LLP, P.O. BOX 55874, BOSTON, MA, 02205, US

(73) Assignee: Worldwide PE Patent Holdco Pty Ltd., Alexandria, VA (US)

(21) Appl. No.: 10/564,273

(22) PCT Filed: Jul. 9, 2004
(86) PCT No.: PCT/AU04/00931

§ 371 Date: Jul. 31, 2007

(30) Foreign Application Priority Data
Jul. 11, 2003 (AU) 2003903597

(51) Int. Cl. A61K 31/138; A61K 31/13; A61K 31/335; A61K 31/38; A61K 31/451; A61K 31/498; A61K 31/55; A61K 9/127; A61K 9/72; A61P 15/12; A61K 9/14; A61K 9/00; A61K 31/5355; A61K 31/497; A61K 31/42; A61K 31/343; A61K 31/135

(52) U.S. Cl. 424/450; 424/400; 424/489; 514/214.02; 514/217; 514/237.8; 514/250; 514/254.05; 514/254.06; 514/321; 514/378; 514/431; 514/450; 514/469; 514/645; 514/646; 514/649; 514/654; 514/657

(57) ABSTRACT

The invention relates to methods and compositions for treating premature ejaculation in a male or prolonging intercourse comprising administering to the male an antidepressant via a route selected from the group consisting of mucosal administration (preferably nasal, buccal or rectal), administration to the lungs (preferably by inhalation), local administration to at least a part of the male genitalia (applied to the penis for example, in the form of a gel) and combinations thereof.

### FIELD OF THE INVENTION

The invention relates to methods and compositions for the treatment of male sexual dysfunction, and particularly to the treatment of premature ejaculation.

### BACKGROUND ART

Mechanical devices have been previously employed in attempts to prevent premature ejaculation. Such devices operate by reducing the stimulation of the penis, but are often awkward and uncomfortable and may not be particularly effective in desensitising the most sensitive part of the penis which is the glans.

Antidepressants have been administered orally, in tablet form, to treat premature ejaculation, although there are significant side effects with this approach. In particular, antidepressants can lead to nausea, vomiting and diziness. Furthermore, their effects are very sensitive to the amount and timing of food and alcohol ingested, as well as the amount of fat on the patient, so their effects can be unpredictable.

Premature ejaculation has been treated in the past by the topical application of compositions containing local anaesthetics, such as lidocaine, to the skin of the penis to reduce sensitivity.

One limitation of such a method of treatment is that the composition must be substantially removed from the skin prior to intercourse to avoid transferring the anaesthetic to a female partner, thereby reducing reduced vaginal sensitivity.

Overapplication of the topical anaesthetic composition is also possible, leading to substantially diminished enjoyment of intercourse by the male.

Any discussion of the prior art throughout the specification should in no way be considered as an admission that such prior art is widely known or forms part of common general knowledge in the field.

It is an object of the present invention to overcome or ameliorate at least one of the abovementioned disadvantages of using conventional anaesthetics.

### DESCRIPTION OF THE INVENTION

According to a first aspect, the invention provides a method of treating premature ejaculation in a male comprising administering to the male an antidepressant via a route selected from the group consisting of i) mucosal administration, ii) administration to the lungs iii) local administration to the male genitalia, and iv) a combination of two or more of mucosal administration, administration to the lungs and local administration to the male genitalia.

Preferably, the mucosal administration is nasal administration although it may also be via other mucosal routes such as buccal administration or rectal administration.

Preferably, administration to the lungs is by way of inhalation.

Preferably, the local administration is directly to the penis.

Specifically, the antidepressant or a combination of antidepressants is administered by one of the abovementioned routes alone or by a combination of mucosal (preferably nasal) and topical (preferably to the penis), or by a combination of mucosal (preferably nasal) and lung (preferably by inhalation), or by a combination of topical preferably to the penis) and lung (preferably by inhalation), or even by a combination of all three, ie a combination of topical (preferably to the penis), mucosal (preferably nasal) and lung (preferably by inhalation) administration.

The most preferred single modes of administration are nasal, to the lungs, and buccal. The most preferred combination modes of administration are combinations of nasal and to the penis, lungs and to the penis, and buccal and to the penis.

For example, the treatment of the present invention maybe administered by way of a nasal spray, an inhaler or a troche, either alone or in combination with a suitable formulation applied to the penis. In combination therapies, the same or different antidepressants may be administered via different routes.

Without wishing to be bound by theory, it is believed the routes of the present invention allow the drugs to bypass the first metabolism of the liver, and cross the blood/brain barrier to act straight away on the brain. This mode of action may serve to explain the small doses used to achieve success, the rapid onset of action and the consistency in dose/response patterns.

The combination of topical (to the penis) and the mucosal routes above appear to result in a synergistic enhancement of the effect in preventing premature ejaculation.

According to a second aspect, the invention provides a method of treating premature ejaculation in a male comprising the step of administering to the male an antidepressant, wherein said antidepressant is administered via a route selected from the group consisting of i) nasal administration, ii) administration to the lungs, iii) buccal administration, iv) administration to the penis and v) a combination of two or more of nasal administration, administration to the lungs, buccal administration and local administration to the penis.

Surprisingly, the present inventor has found that antidepressants administered by these routes result in a sufficient degree of anaesthesia to the penis to overcome premature ejaculation.

The term antidepressant as used herein refers to any substance used in the treatment of clinical depression. All antidepressants are believed to be suitable for use in the present invention.

In one preferred embodiment, the antidepressant is a selective serotonin reuptake inhibitor (known as an SSRI).

In alternative preferred embodiments the antidepressant is a bicyclic, tricyclic or tetracyclic antidepressant.

In yet another alternative preferred embodiment, the antidepressant is a monoamine oxidase inhibitor.

In further alternative preferred embodiments, the antidepressant may be selected from one of the following classes: Serotonin Norepinephrine Reuptake Inhibitors, Norepinephrine Dopamine Reuptake Inhibitors, Serotonin Antagonist and Reuptake Inhibitors, Norepinephrine Antagonist/Serotonin Antagonists, Monoamine Oxidases (MAO) Inhibitors, synthetically derived phenylpiperazine antidepressants, antagonists of central L2-α2 auto and heteroadrenoceptors.

As mentioned, the inventor has found that an antidepressant agent, that is an agent that is more typically used for the treatment of clinical depression, is capable of delaying ejaculation when administered by nasal/lung/buccal or a combination of these, or in combination with administration to the penis, while minimising side effects typical of such antidepressants such as vomiting, nausea and dizziness.

Further, the agent can be administered at a fraction of the dose than is usually used to treat depression, with rapid onset of action and with a high degree of predictability.

Importantly, for the treatment of premature ejaculation, the inventor has found that when topically applied to at least a portion of the skin of the male genitalia, for example, when applied to the penis, an antidepressant agent will induce a sufficient degree of anaesthesia to provide an improvement to the condition of premature ejaculation.

Even more surprisingly, it has been found that administering antidepressants mucosally, preferably nasally, or by administration to the lungs, delays ejaculation during intercourse.

A combination of mucosal, preferably nasal, and local administration has been found to be extremely useful in preventing premature ejaculation and prolonging sexual intercourse.

Alternatively, a combination of administration to the lungs, and local administration has been found to be extremely useful in preventing premature ejaculation and prolonging sexual intercourse.

A further combination of routes of administration, namely a combination of mucosal, preferably nasal, and administration to the lungs has also been found to be valuable in preventing premature ejaculation and prolonging sexual intercourse.

One or more antidepressants may be used in combination, and they may be used alone or with one or more carriers for facilitating the application of the antidepressant agent to the skin or too the mucosa.

If a combination of two or more routes selected from mucosal administration, administration to the lungs and local administration is used, then the same antidepressant or antidepressant combination may be administered by the two or more different routes, or different antidepressants or combinations of antidepressants may be administered by the two or more different routes.

According to a third aspect, the invention provides a composition for the treatment of premature ejaculation, said composition comprising an antidepressant formulated for mucosal administration. More preferably, the antidepressant is formulated for nasal administration.

According to a fourth aspect, the invention provides a composition for the treatment of premature ejaculation, said composition comprising an antidepressant formulated for administration to the lungs. Most preferably, administration to the lungs is by inhalation.

According to a fifth aspect, the invention provides a composition including an antidepressant formulated for local administration to the male genitalia.

According to a sixth aspect, the invention provides a kit, said kit comprising an antidepressant formulated for nasal administration and an antidepressant formulated for topical application. Preferably, the antidepressants are selected such that a synergistic interaction occurs when there is a combination of nasal and local administration.

According to a seventh aspect, the invention provides a kit, said kit comprising an antidepressant formulated for administration to the lungs and an antidepressant formulated for topical application. Preferably, the antidepressants are selected such that a synergistic interaction occurs when there is a combination of lung and local administration.

According to a eighth aspect, the invention provides a kit, said kit comprising an antidepressant formulated for nasal administration and an antidepressant formulated for application to the lungs. Preferably, the antidepressants are selected such that a synergistic interaction occurs when there is a combination of nasal and lung administration.

According to a ninth aspect, the invention provides a kit, said kit comprising an antidepressant formulated for nasal administration, an antidepressant formulated for application to the lungs and an antidepressant formulated for topical administration. Preferably, the antidepressants are selected such that a synergistic interaction.

According to a tenth aspect the invention provides a method of prolonging sexual intercourse involving a male, said method including the step of administering to said male prior to intercourse an amount of an antidepressant effective to delay ejaculation; and wherein said antidepressant is administered via a route selected from the group consisting of: mucosal administration, administration to the lungs, topical administration to the male genitalia, and a combination of two or more of mucosal administration, administration to the lungs or topical administration to the male genitalia.

According to an eleventh aspect the invention provides a method of prolonging sexual intercourse involving a male and a female, said method including the step of administering to said male prior to intercourse an amount of an antidepressant effective to delay ejaculation without anaesthetising the female genitalia; and wherein said antidepressant is administered via a route selected from the group consisting of: mucosal administration, administration to the lungs, topical administration to the male genitalia, and a combination of two or more of mucosal administration, administration to the lungs or topical administration to the male genitalia.

### Antidepressants

As mentioned, any antidepressant is suitable for use in the present invention. The antidepressants may be selected from:
Selective Serotonin Reuptake Inhibitors (SSRI)
Serotonin Norepinephrine Reuptake Inhibitors, (including bicyclic, tricyclic and tetracyclic antidepressants)
Norepinephrine Dopamine Reuptake Inhibitors
Serotonin Antagonist and Reuptake Inhibitors
Norepinephrine Antagonist/Serotonin Antagonists
Monoamine Oxidases (MAO) Inhibitors
Synthetically derived phenylpiperazine antidepressants
antagonists of central L2-α2 auto and heteroadrenoceptors

Some specific non-limiting examples of antidepressants include, but are not limited to paroxetine, fluoxoetine and sertraline. Paroxetine may be formulated as a hydrochloride (such as Aropax) or in the form of other salts or in combination with other bases such as mesylates. Other suitable antidepressants for use in the present invention include citalopram hydrobromide (Cipramil), fluoxetine (Prozac), fluvoxamine (Luvox), sertraline (Zoloft), nortriptyline hydrochloride (Allegron), clomipramine hydrochloride (Anafranil), dothiepin hydrochloride (Prothiaden), Imipramine hydrochloride (Tofranil), mianserin hydrochloride (Tolvon), amitriptyline hydrochloride (Tryptanol), phenelzine sulphate (Nardil), tranylcypromine sulphate (such as Parnate), isocarboxazid (Marplan), moclobemide (Aurorix), serotonin and/or adrenalin update inhibitors such as venlafaxine (Efexor), nefazodone hydrochloride (Serzone), trazodone (Desyrel), bupropion (Zyban), mirtazapine (Remeron), doxepin hydrochloride (Sinequan) and trimipramine (Surmontil).

Preferably, the antidepressant is administered in an amount of between 0.1 and 1000 mg per dose, depending upon the nature of active ingredient used and the severity of the patients problems, as well as other factors such as patient size. More preferably, the dosage will be between 1 and 100 mg antidepressant, even more preferably between 5 and 25 mg antidepressant. The exact dosage will be readily determined by a trained clinician.

Topical Formulations

For topically administered antidepressants, administration is typically by way of application either to the skin of the region of the male genitalia as a composition that may also include a carrier for facilitating the application of the antidepressant agent to the skin.

Compositions may be applied to the male genitalia in the form of a gel, although they may also be formulated as a lotion or a powder. It will be understood by those skilled in the art that the particular form of the composition is not important, provided that by contacting the composition with the skin in the male genital region, the antidepressant agent is permitted to induce anaesthesia that that region.

The composition may be applied to any part of the male genitalia, such as the penis only, or also to the scrotum or surrounding regions such as the perineum. Most usually, a topical composition comprising an antidepressant is applied directly to the penis.

The topical composition is typically applied by massaging into the skin for about one minute, preferably about 30 to 60 minutes prior to intercourse.

Preferably, the concentration of the antidepressant in a composition is between about 1 to 10% by weight, preferably about 3 to 6% by weight of the topical composition.

Preferably, the antidepressant in the topical formulation is present in an amount of between 0.1 and 1000 mg per dose, depending upon the active ingredient used. More preferably, the dosage will be between 1 and 100 mg antidepressant, even more preferably between 5 and 25 mg antidepressant.

It will be understood by those skilled in the art that more than one type of agent described above could be used in the composition of the invention, provided that the composition is capable of introducing anaesthesia at a region of male genitalia either to which the composition is applied, or to the appropriate regions by means of mucosal administration.

The composition may be provided in the form of a gel or lotion, or alternatively, it may be provided as a powder which in use can be hydrated to form a gel or lotion suitable for application to a region of male genitalia.

Most typically, the composition is applied to the skin of the male genitalia as a gel. A typical gel for facilitating application of the antidepressant is a hydrogel such as hydroxypropylmethylcellulose (Methocell EM4), or an acrylic acid polymer such as Carbopol 943P. The polymer in the composition is present in the range of about 0.1 to about 5 wt %. The carrier is typically water soluble, non-irritating and does not sensitise the skin. It is desirable if the carrier imparts a semi soft creamlike consistency to the composition.

The topical compositions may further comprise an enhancer agent for enhancing the absorption of the antidepressant agent through the skin. Examples of enhancer agent include cyclodextrins such as α-, β-, and γ-cyclodextrin and 2-methylcyclodextrin. Hydroxypropyl-β-cyclodextrin HPBCD is particularly advantageous as compositions comprising HPBCD are suitable for use with diabetic men. HPBCD is a cyclic polymer having a ring shaped molecular structure including an inner cavity. It is understood that an inclusion compound is formed with HPBCD which makes the antidepressant more readily absorbed by the skin. The weight percent of the HPBCD in the composition is preferably in the range of about 1 to 10%.

HPBCD is a commercially available compound derived from β-cyclodextrin a condensation with propylene oxide to provide the corresponding hydroxypropyl derivative having a degree of substitution of up to about 15 or higher. A degree of substitution of about 5 to about 7 is preferred for compositions of the present invention.

The antidepressant agent and the HPBCD are typically present in the composition in a molar ratio of about 1:0.8 to 1:1.4 respectively. Preferably, the antidepressant agent and the HPBCD are present in a ratio of about 1:1.

The pHs of the compositions in the present invention are preferably in the range of about 2 to about 8, and more preferably, around 7.4. The pH can be adjusted by any physiologically suitable agent such as amoniumhydroxide or sodium hydroxide.

Other components of the composition may include water, monohydric and polyhydric alcohols such as ethanol, polyethylene glycol, propylene glycol and DMSO. The amount of water in the composition is typically in the range of about 20 to about 60%, and alcohols typically about 80% to about 40%. The ethanol and the propylene glycol are preferably present in a relative weight ratio of about 3:1 to about 0:1.

Preferably, the composition need not be removed after application.

### Mucosal Formulations

For mucosally administered antidepressants, administration is via application to the mucosa of said male as a composition that may include a carrier for facilitating the application of the antidepressant agent to the mucosa.

Similarly, it will be understood that the compositions for mucosal administration may be in many forms. Most typically, the invention is formulated as a spray for nasal administration, although it may be formulated for buccal administration in the form of a troche or in the form of a suppository for rectal administration. Those skilled in the art will appreciate that the important consideration is to select the antidepressant, the dosage and the dosage form in combination to provide a sufficient quantity of antidepressant is present to induce a degree of anaesthesia in the male genitalia to prevent premature ejaculation.

Nasal formulations include gels, suspension, liposomal dispersions, emulsions or microemulsions and may be any combination of aqueous and non-aqueous components. Alternatively, the nasal formulations may be in powdered form, such as microspheres, liposomes, coated microspheres (for example, such as those with a cellulose or polysaccharide coating).

The nasal formulations of the present invention may include conventional additives and excipients, such as buffers, thickening agents, soothing agents, sweeteners and membrane conditioners or transport agents, antioxidants, preservatives, penetrating agents and other carriers which will be known by those skilled in the art. It is preferably dispensed via a metered spray vessel. Administering the dose in a metered fashion enables a use of a defined quantity of the active ingredient involved.

Nasal formulations may typically include water, polyethylene glycols (various pharmaceutically acceptable PEG's,) glycerine, DMSO, ascorbic acid or ascorbate salts or bisulfites.

Preferably, the antidepressant in the nasal formulation is present in an amount of between 0.1 and 1000 mg per dose, depending upon the active ingredient used. More preferably, the dosage will be between 1 and 100 mg antidepressant, even more preferably between 5 and 25 mg antidepressant.

Preferably, the dosage is taken nasally just prior to intercourse, between 10 and 30 minutes prior to intercourse, most preferably around 20 minutes prior to intercourse. The nasal composition may be administered by means of one or two metered doses shortly before intercourse, or by way of a troche or suppository which are administered slightly further ahead of time. A typical nasal spray of the present invention contains antidepressant (name) such that a single dose typically delivers from 2 to 50 mg antidepressant (name). The volume of one actuation of a metered dose is generally 20 to 500 microlitres.

| | |
|---|---|
| antidepressant (name) | 100 mg |
| Antioxidant | 1% |
| Preservative | 0.5% |
| Dimethyl sulfoxide | 0.02% |
| Purified Water | to 100. |

### Formulation for Application to the Lungs

Any conventional formulation for administration to the lungs may be used. Preferably, this is via inhalation. A propellant is preferably also included in such formulations.

The drug may be delivered in the form of, for example a dry powder, a micronized drug suspended in a liquefied propellant, or a drug dissolved, either alone or by way of a cosolvent, in a liquefied propellant.

Preferably, the particle size of the dry material is less than 10 microns, and preferably less than about 5 microns

Aerosols propellants include any agents suitable for medical use provided they are compatible with the active. They may be, for example, CFC (chlorofluorocarbon) or HFA (hydrofluoroalkane) propellants.

All types of nebuliser may be used—pressure, powder, metered powder or ultrasonic.

Where the active antidepressant is dissolved, a cosolvent may be added. A suitable cosolvent is, for example, ethanol.

Other ingredients, may be added to the formulations. These may include, for instance, surface active agents (surfactants). Any suitable Surface active agent may be used. These may include, for example, oleic acid, sorbitan trioleate and lecithin.

Preferably, the antidepressant is administered in an amount of between 0.1 and 1000 mg per metered dose. More preferably, the dosage will be between 1 and 100 mg antidepressant, even more preferably between 5 and 25 mg antidepressant.

### Results

A range of commonly available antidepressants were prepared and administered according to the present invention as described above.

All three routes of administration, oral, nasal and a combination of topical and nasal were found to produce a reduction in premature ejaculation as a whole. Those subjects taking the antidepressants by both the nasal and the topically administered formulations noted particularly good results.

For those patients taking the antidepressants by both routes, it may be advantageous if the antidepressants were selected from different groups, eg if one was an SSRI and the other was a MAO inhibitor, for example.

Antidepressants were administered to a large number of subjects in accordance with the method of the present invention. The study involved in excess of 200 patients in each treatment group. All subjects reported experiencing premature ejaculation prior to commencing the study. All medicaments were self administered. Topical administration was to the skin of the glans. Paroxetine, Fluoxetine and Sertraline were administered randomly via a mixture of routes.

| | Number subjects | |
|---|---|---|
| Administration | initially experiencing | |
| route: | PE | Overall Result |
| Nasal | >200 | + |
| Buccal | >200 | + |
| Pulmonary | >200 | + |
| Nasal + Topical | >200 | ++ |

Assessment was made by asking patients whether they were satisfied with the result. Almost all subjects reported an improvement in time to ejaculation. A combination of nasal and topical seemed to be most satisfactory, in providing the best result to the largest number of subjects.

While the invention is described with reference to specific embodiments, it will be understood by those skilled in the art that that variations and modifications may be made without departing from the inventive concept disclosed herein.

1-61. (canceled)

62. A method of treating premature ejaculation in a male comprising administering to the male an antidepressant via a route selected from the group consisting of:
i) mucosal administration;
ii) administration to the lungs
iii) local administration to at least a part of the male genitalia and
iv) a combination of two or more of mucosal administration, administration to the lungs and local administration to at least a part of the male genitalia.

63. A method according to claim 1 wherein the antidepressant is administered prior to or during sexual activity.

64. The method according to claim 1 wherein the mucosal administration is by nasal administration, buccal administration, rectal administration or combinations thereof.

65. The method according to claim 1 wherein administration to the lungs is by way of inhalation.

66. The method according to claim 1 wherein local administration is directly to the penis.

67. A method according claim 1 wherein the antidepressant is administered by a combination of mucosal administration and local administration.

68. The method according to claim 1 wherein local administration is topical administration to the penis and administration to the lungs is nasal administration.

69. The method according to claim 1 wherein the antidepressant is administered in an amount of between 0.1 and 1000 mg per dose.

70. The method according to claim 1 wherein a single antidepressant or single combination of antidepressants is administered by two or more routes selected from the mucosal administration, administration to the lungs and local administration to at least a part of the male genitalia.

71. The method according to claim 1 wherein two or more different antidepressants or two or more different combinations of antidepressants are administered by two or more routes selected from the mucosal administration, administration to the lungs and local administration to at least a part of the male genitalia.

72. The method according to claim 10 wherein the antidepressant combination includes serotonin reuptake inhibitor (SSRIs) and a monoamine oxidase inhibitor.

73. The method according to claim 1 wherein the antidepressant is massaged into the skin of the male genitalia prior to intercourse for about one minute.

74. The method according to claim 12 wherein the antidepressant is massaged into the skin of the male genitalia about 30 to 60 minutes prior to intercourse.

75. The method according to claim 1 wherein the antidepressant is taken nasally within 30 minutes prior to intercourse.

76. A composition for the treatment of premature ejaculation, said composition comprising an antidepressant formulated for mucosal administration, administration to the lungs and/or local administration to at least part of the male genitalia.

77. The composition according to claim 15 wherein said antidepressant is selected from serotonin reuptake inhibitors, serotonin norepinephrine reuptake inhibitors, monoamine oxidase inhibitors, norepinephrine dopamine reuptake inhibitors, serotonin antagonist and reuptake inhibitors, norepinephrine antagonist/serotonin antagonists, synthetically derived phenylpiperazine antidepressants, antagonists of central L2-α2 auto and heteroadrenoceptors, and mixtures thereof.

78. The composition according to claim 15 wherein said antidepressant is a mixture of a serotonin reuptake inhibitor and a monoamine oxidase inhibitor.

79. The composition according to claim 17 wherein said serotonin norepinephrine reuptake inhibitor is a bicyclic, tricyclic or tetracyclic antidepressant.

80. The composition according to claim 15 wherein said antidepressant is selected from paroxetine, fluoxoetine, sertraline, citalopram hydrobromide, fluvoxamine, sertraline, nortriptyline hydrochloride, clomipramine hydrochloride, dothiepin hydrochloride, Imipramine hydrochloride, mianserin hydrochloride, amitriptyline hydrochloride, phenelzine sulphate, tranylcypromine sulphate, isocarboxazid, moclobemide, serotonin and/or adrenalin update inhibitors such as venlafaxine, nefazodone hydrochloride, trazodone, bupropion, mirtazapine, doxepin hydrochloride, trimipramine or mixtures thereof.

81. The composition according to claim 15 formulated for local administration directly to the penis.

82. The composition according to claim 15 further including an enhancer agent selected from cyclodextrin, water, a monohydric alcohol, a polyhydric alcohol

83. The composition according to claim 15 comprising:
about 20 to about 60% water;
about 80% to about 40% alcohols
1 to 10% Hydroxypropyl-β-cyclodextrin (HPBCD) and wherein antidepressant agent and the HPBCD are typically present in the composition in a molar ratio of about 1:0.8 to 1:1.4 respectively.

84. The composition according to claim 15 wherein the concentration of the antidepressant is between about 1 to 10% by weight of said composition.

85. A kit comprising at least two of: an antidepressant formulated for nasal administration; an antidepressant formulated for local administration to at least part of the male genitalia and an antidepressant formulated for administration to the lungs.
